(19) United States
(12) Patent Application Publication
(10) Pub. No.: US 2007/0197967 A1
(43) Pub. Date: Aug. 23, 2007

(54) Safety syringe

(75) Inventors: Chien-Yuan Lee, Tamsui Town (TW); Chih-Cheng Kao, Taipei (TW)

Correspondence Address: COOPER & DUNHAM, LLP, 1185 AVENUE OF THE AMERICAS, NEW YORK, NY, 10036, US

(73) Assignee: Linfair Engineering (HK) Co., Ltd.

(21) Appl. No.: 11/358,329

(22) Filed: Feb. 21, 2006

(51) Int. Cl. A61M 5/00

(52) U.S. Cl. 604/110

(57) ABSTRACT

A safety syringe has a syringe body, a casing, a connector and a needle hub with a needle. The syringe body has a front end and a neck. The neck is formed on the front end. The casing is mounted around the syringe body and has a front end, an inner surface, and a fix unit. The fix unit is formed on the inner surface. The connector is mounted around the neck and has a rear tube and a notch. The rear tube is mounted around the neck. The notch is formed around the rear tube. The needle hub is mounted around the connector. A user pushes the rear end of the casing to the front end of the syringe body so the needle hub is mounted in the casing. Breaking the notch, the casing is detached from the syringe body and holds the needle inside.

### BACKGROUND OF THE INVENTION

1. Field of the Invention

The present invention relates to a syringe, and especially to a safety syringe for protecting a user from being pricked with a used needle.

2. Description of the Related Art

With modern developments in medical technology, people understand clearer about routes of infection. One of the infection routes is blood contact. If a person is pricked with a used needle, the used needle can transfer diseases to the person. Therefore, the used needle must be disposed carefully. However, a user is pricked easily with the used needle during inserting the used needle into a cap.

### SUMMARY OF THE INVENTION

The objective of the present invention is to provide a safety syringe that can avoid pricking with a used needle during the used needle inserts into a cap.

To achieve the foregoing objective, a safety syringe has a syringe body, a casing, a plunger, a connector and a needle hub with a needle. The syringe body has a front end, a rear end and a neck. The neck is formed on the front end. The casing is mounted around the syringe body and has a front end, a rear end, an inner surface, and a fix unit. The fix unit is formed on the inner surface near the rear end. The plunger is mounted in the syringe body. The connector is mounted around the neck and has a middle ring, a front tube, a rear tube and a notch. The middle ring is formed between the front tube and the rear tube. The rear tube is mounted around the neck and has an outer surface. The notch is formed around the outer surface of the rear tube. The needle hub is mounted around the front tube. After injecting, a user pushes the rear end of the casing from the rear end to the front end of the syringe body so the needle hub with the needle is mounted in the casing and the connector engages with the fix unit. Then breaking the notch, the casing is detached from the syringe body and holds the needle inside.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a first embodiment of a safety syringe in accordance with the present invention;

FIG. 2 is an explored perspective view of the first embodiment of the safety syringe in FIG. 1;

FIG. 3 is a perspective view of a connector of the safety syringe in FIG. 1;

FIG. 4 is a side view in partial section of the first embodiment of the safety syringe in FIG. 1;

FIG. 5 is a side view in partial section of the first embodiment of the safety syringe in FIG. 1 showing that the needle is inserted into a casing;

FIG. 6 is an operational side view in partial section of the first embodiment of the safety syringe in FIG. 1 showing that the safety syringe is broken at a notch;

FIG. 7 is a perspective view of a second embodiment of a safety syringe in accordance with the present invention;

FIG. 8 is a perspective view of a connector of the safety syringe in FIG. 7;

FIG. 9 is a side view in partial section of the second embodiment of the safety syringe in FIG. 7 showing that the needle is inserted into the casing;

FIG. 10 is an operational side view in partial section of the second embodiment of the safety syringe in FIG. 7 showing that the safety syringe is broken at a notch;

FIG. 11 is an operational side view in partial section of the second embodiment of the safety syringe in FIG. 7 showing that the syringe body is detached from the casing and the needle hub with the needle is kept inside the casing;

FIG. 12 is an operational side view in partial section of a third embodiment of a safety syringe in accordance with the present invention;

FIG. 13 is a perspective view of a connector of the safety syringe in FIG. 12;

FIG. 14 is an operational side view in partial section of a forth embodiment of a safety syringe in accordance with the present invention; and

FIG. 15 is a side view in partial section of a connector of the safety syringe in FIG. 14.

### DETAILED DESCRIPTION OF THE PREFERRED EMBODIMENTS

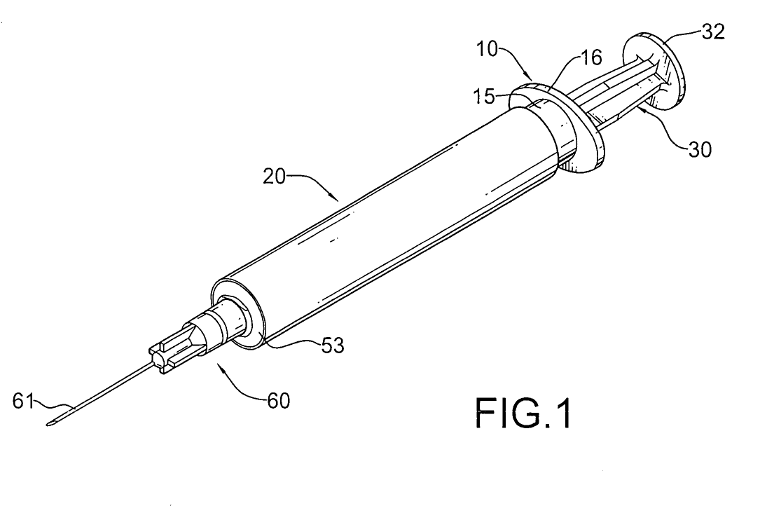

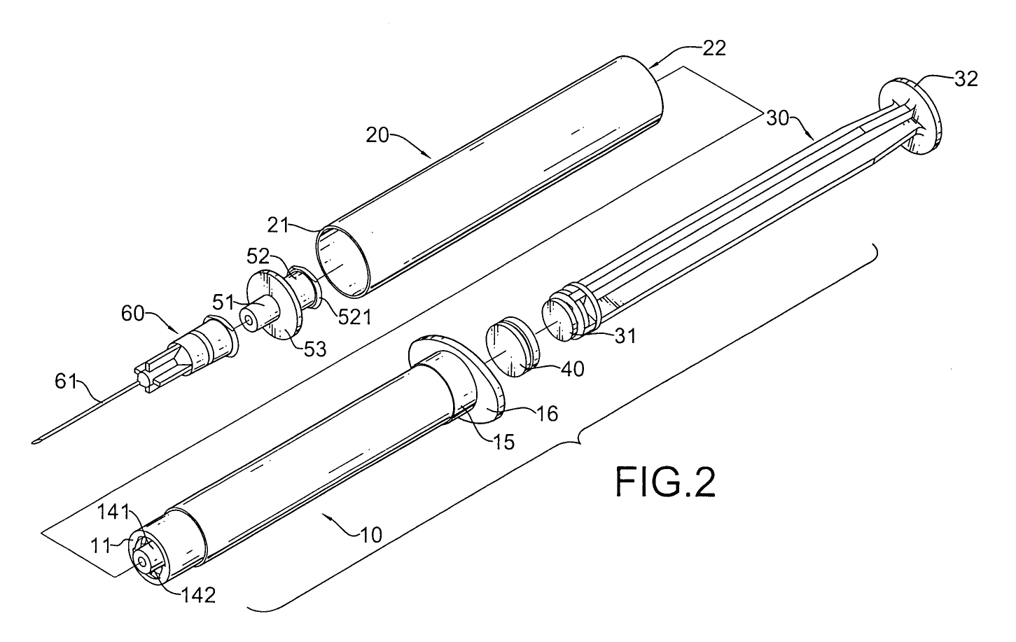

With reference to FIGS. 1 to 3, a safety syringe in accordance with the present invention comprises a syringe body (10), a casing (20), a plunger (30), a connector (50), a needle hub (60) and an optional plug (40).

The syringe body (10) is tubular and comprises a front end (11), a rear end (12), an outer surface, an outer diameter, a neck (141), an outer tube (142), an optional annular protrusion (15), at least one optional projection (16). The neck (141) is formed on the front end (11). The outer tube (142) is formed around the neck (141), extends from the syringe body (10), is coaxial with the neck (141) and further comprises an inner thread. The annular protrusion (15) is formed on the outer surface near the rear end (12). The at least one projection (16) is respectively formed on the rear end (12) and opposite to each other to allow a user to hold the syringe body (10) during injection.

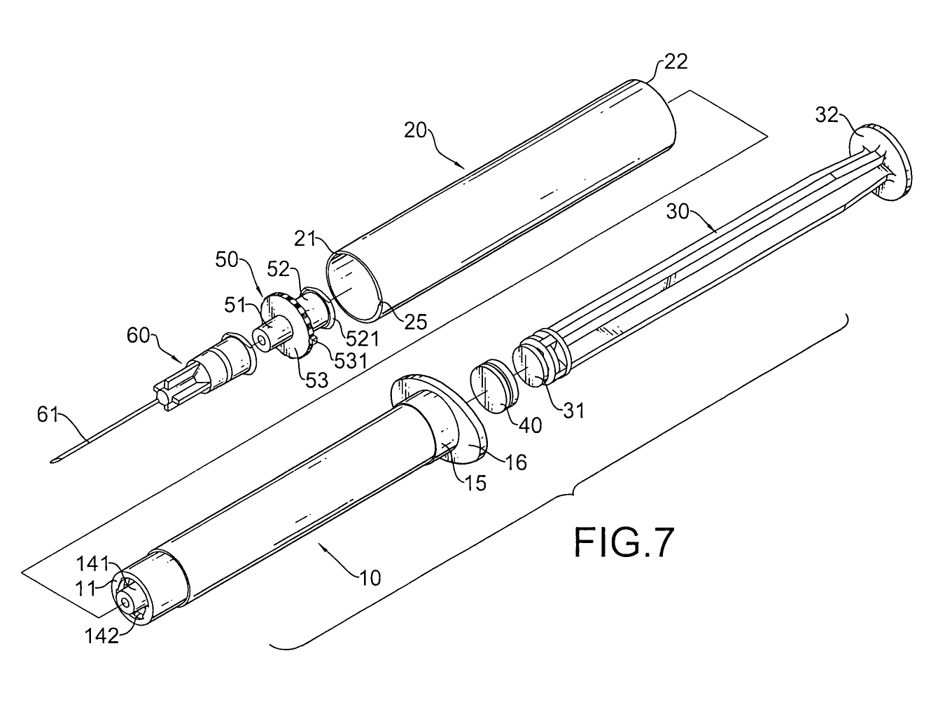

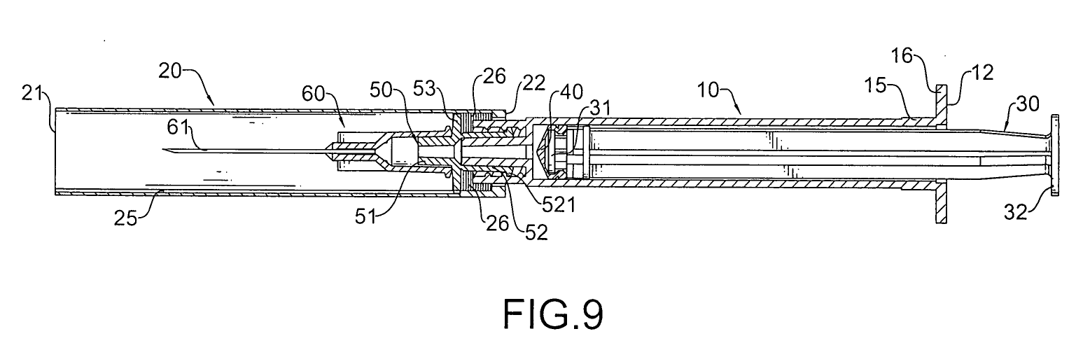

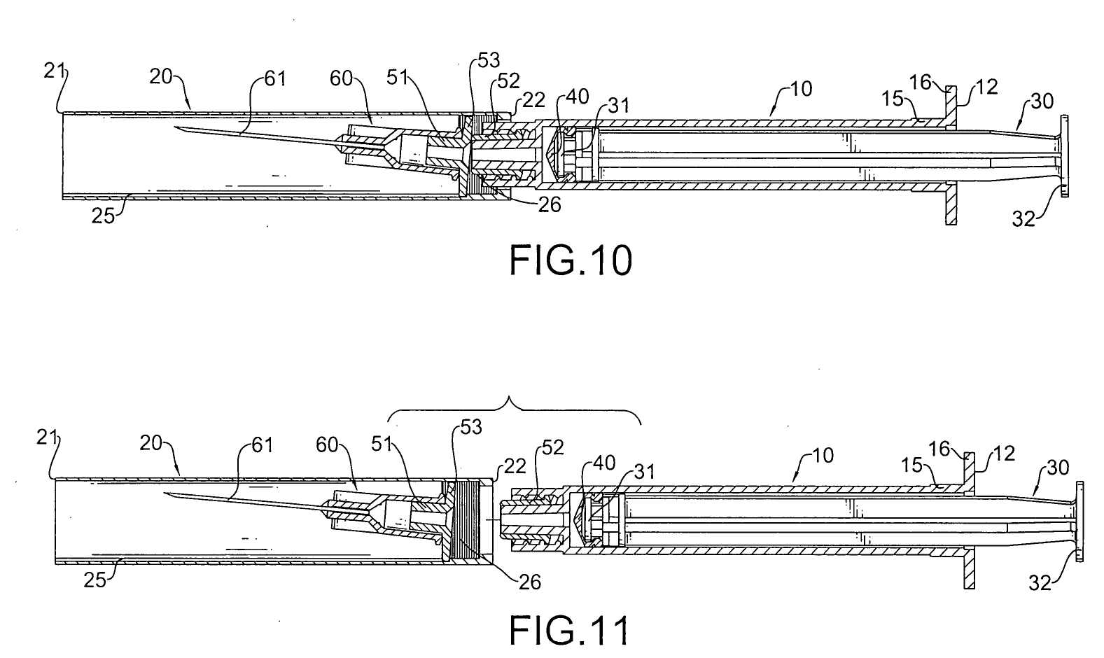

The casing (20) is tubular, is mounted around the syringe body (10) and comprises a front end (21), a rear end (22), an inner diameter, an inner surface, a fix unit and an optional guide slot (25) as shown in FIG. 7. The rear end (22) attaches with the annular protrusion (15). The inner diameter of the casing (20) is larger than the outer diameter of the syringe body (10). The fix unit is formed on the inner surface near the rear end (22) and may comprise a slope (23) and an annular recess (24) or may comprise at least one resilient tab and a protruding ring or may comprise multiple protruding rings (26) as shown in FIG. 9. The slope (23) is formed on the inner surface. The annular recess (24) is formed in the slope (23).

The plunger (30) is mounted in the syringe body (10), and comprises a front end, a rear end, a base (31) and a push head (32). The base (31) is formed on the front end. The push head (32) is formed on the rear end. Pushing the push head (32) ejects the medical solution from the syringe body (10).

The plug (40) is mounted around the base (31) of the plunger (30).

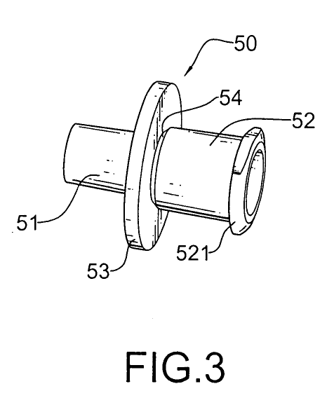

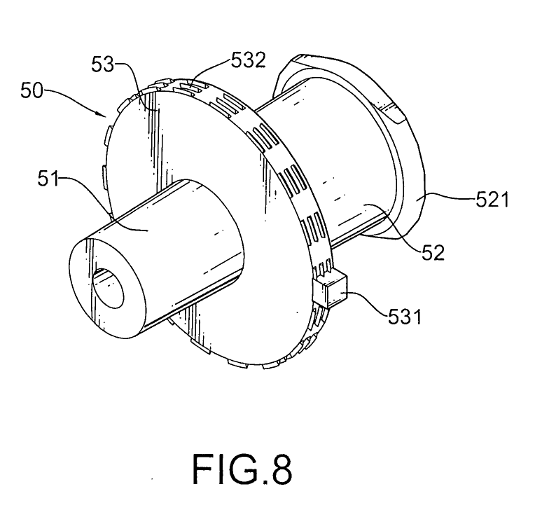

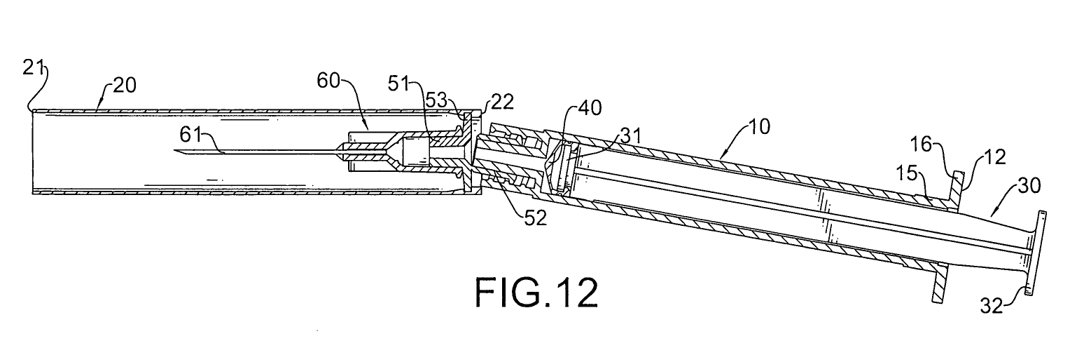

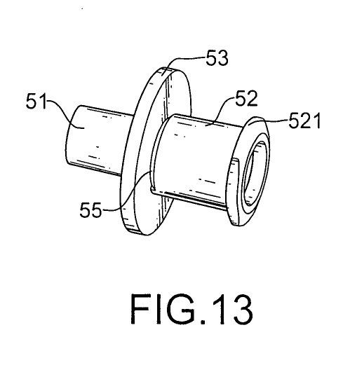

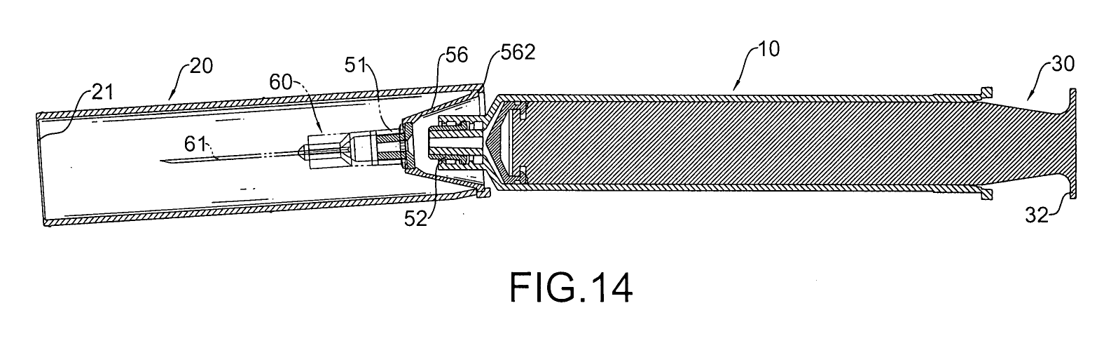

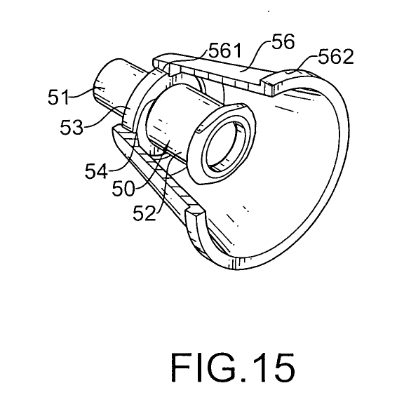

With further reference to FIGS. 3, 8 and 13 to 15, the connector (50) is tubular and hollow, is mounted around the neck (141) and comprises a front tube (51), a rear tube (52), a middle ring (53), a notch (54, 55) and an optional fix taper (56). The middle ring (53) is formed between the front tube (51) and the rear tube (52) and further comprises an outer circumference, a guide protrusion (531) and multiple limit protrusions (532). The guide protrusion (531) is formed on and protrudes from the outer circumference of the middle ring (53), slides in the guide slot (25) and is higher than the limit protrusions (532). The limit protrusions (532) are separately formed on and protrudes from the outer circumference of the middle ring (53) in equal interval. The limit protrusions (532) attach to the protruding rings (26), when pushing the rear end (22) of the casing (20) from the rear end (12) to the front end (11) of the syringe body (10). The rear tube (52) is mounted around the neck (141) on the syringe body (10) and comprises an outer surface, a proximal end, a distal end and an optional rib (521). The rib (521) is formed around the outer surface on the distal end and attaches the inner thread in the outer tube (142). The notch (54, 55) is formed around the outer surface of the rear tube (52) and may be formed incompletely around the rear tube (52), such as the notch (55) as shown in FIG. 13, so the casing (20) does not detach completely from the syringe body (10) after breaking the notch (55) as shown in FIG. 12. Preferably, the notch (54, 55) is formed around the outer surface of the rear tube near the proximal end. The notch (54, 55) can be annular or a spiral. The fix taper (56) is mounted around the connector (50) and comprises a front end, a rear end, an outer surface, an inner surface, a groove (561) and a circumference protrusion (562). The groove (561) is mounted around the middle ring (53) and is formed around the inner surface on the front end. The circumference protrusion (562) is formed around the outer surface on the rear end and attaches with the annular recess (24) when the casing (20) is moved to the front end of the syringe body (10).

The needle hub (60) is mounted around the front tube (51) and comprises a needle (61). The needle hub (60) may screw, clamp, glue to, weld or stick on the front tube (51).

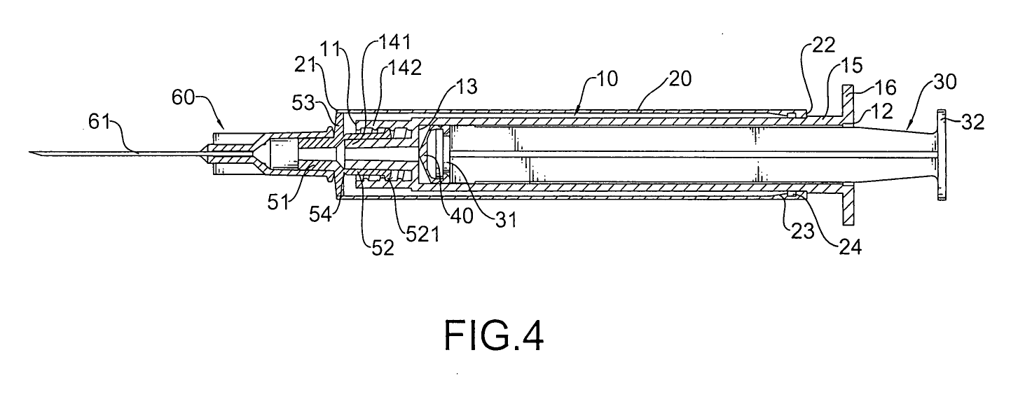

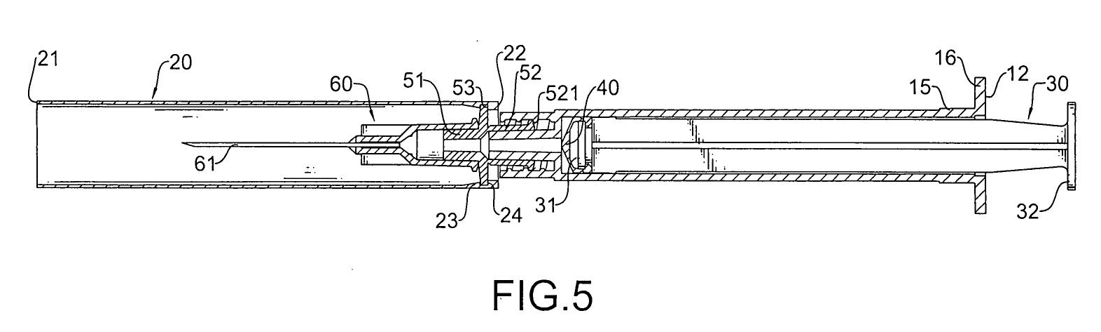

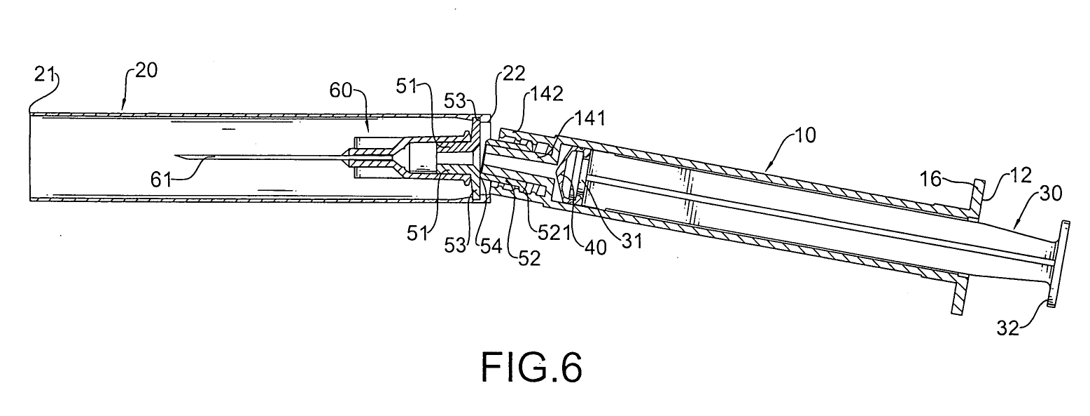

With reference to FIG. 4 to 6, 9 to 12 and 14, after injecting, a user pushes the rear end (22) of the casing (20) from the rear end (12) to the front end (11) of the syringe body (10). The middle ring (53) or the circumference protrusion (562) of the connector (50) will be wedged in the annular recess (24). Then, the user can hold the casing (20) in one hand and hold the syringe body (10) in the other to break the syringe body (10) at the notch (54, 55) so the casing (20) is detached from the syringe body (10) and the needle hub (60) with the needle (61) is kept inside the casing (20). Because the middle ring (53) or the circumference protrusion (562) of the connector (50) is wedged in the annular recess (24) and the notch (54, 55) is formed around the outer surface of the rear tube (52) near the proximal end, the needle hub (60) with the needle (61) is kept from being escaped from the casing (20). Therefore, the user will not be pricked by a used needle (61).

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A safety syringe comprising:
a syringe body being tubular and comprising
a front end;
a rear end;
an outer surface;
an outer diameter;
a neck formed on the front end; and
an outer tube formed around the neck, extending from the syringe body,
and being coaxial with the neck;
a casing being tubular, mounted around the syringe body and comprising
a front end;
a rear end;
an inner diameter;
an inner surface; and
a fix unit formed on the inner surface near the rear end;
a plunger slidably mounted in the syringe body and comprising
a front end;
a rear end;
a base formed on the front end; and
a push head formed on the rear head;
a connector being tubular and hollow, mounted around the neck and comprising
a front tube;
a rear tube mounted around the neck on the syringe body and comprising
an outer surface;
a proximal end; and
a distal end;
a middle ring formed between the front tube and the rear tube; and
a notch formed around the outer surface of the rear tube;
a needle hub mounted around the front tube and comprising a needle.

2. The safety syringe as claimed in claim 1, wherein the inner diameter of the casing is larger than the outer diameter of the syringe body.

3. The safety syringe as claimed in claim 1, wherein the fix unit comprises
a slope formed on the inner surface of the casing near the rear end; and
an annular recess formed in the slope.

4. The safety syringe as claimed in claim 1, wherein
the middle ring comprises
an outer circumference; and
a guide protrusion formed on and protruding from the outer circumference; and
the casing comprises
a guide slot corresponding to and slidably engaging with the guide protrusion.

5. The safety syringe as claimed in claim 4, wherein
the casing comprise multiple protruding rings formed on the inner surface near the rear end; and
the middle ring comprises
multiple limit protrusions separately formed on and protruding from the outer circumference in equal interval and engaged with the protruding rings when the casing is moved to the front end of the syringe body.

6. The safety syringe as claimed in claim 1, wherein the notch is formed around the outer surface of the rear tube near the proximal end.

7. The safety syringe as claimed in claim 1, wherein the connector further comprises a fix taper mounted around the connector and comprising
a front end;
a rear end;
an outer surface;
an inner surface;
a groove mounted around the middle ring and formed around the inner surface on the front end; and
a circumference protrusion formed around the outer surface on the rear end.

8. The safety syringe as claimed in claim 1, wherein the notch is annular.

9. The safety syringe as claimed in claim 1, wherein the notch is formed incompletely around the rear tube.

10. The safety syringe as claimed in claim 1, wherein
the outer tube of the syringe body further comprises an inner thread; and
the rear tube of the connector further comprises a rib formed around the outer surface on the distal end and engaging with the inner thread of the outer tube.

11. The safety syringe as claimed in claim 1, wherein the safety syringe further comprises a plug is mounted around the base of the plunger.

12. The safety syringe as claimed in claim 1, wherein the syringe body further comprises
an annular protrusion formed on the outer surface near the rear end; and
at least one projection respectively formed on the rear end and opposite to each other.
